(19) United States
(12) Patent Application Publication
(10) Pub. No.: US 2022/0243239 A1
(43) Pub. Date: Aug. 4, 2022

(54) METHOD FOR SYNTHESIZING LACTO-N-BIOSE

(71) Applicant: SHANDONG HENGLU BIOTECH CO., LTD., Jinan, Shandong (CN)

(72) Inventors: Xu FANG, Yantai (CN); Zhiqiang DU, Yantai (CN)

(73) Assignee: SHANDONG HENGLU BIOTECH CO., LTD., Jinan, Shandong (CN)

(21) Appl. No.: 17/271,054

(22) PCT Filed: Apr. 3, 2020
(86) PCT No.: PCT/CN2020/083230

§ 371 Date: Feb. 24, 2021

(30) Foreign Application Priority Data
Oct. 31, 2019 (CN) 2019110555165

(51) Int. Cl. C12P 19/12; C12N 9/12; C12N 9/24; C12P 19/26

(52) U.S. Cl. CPC C12P 19/12; C12N 9/1205; C12P 19/26; C12N 9/2402; C12Y 302/0114; C12Y 207/01006

(57) ABSTRACT

A method for synthesizing lacto-N-biose and belongs to the technical field of bioengineering and oligosaccharide synthesis. A multi-enzyme catalytic system with good biological safety and wide application, and an ATP regeneration cycle system is introduced into a multi-enzyme reaction system, so that the synthesis of lacto-N-biose and the utilization rate of substrates are improved. A novel lacto-N-biose synthetic route lays a foundation for large-scale industrial production of lacto-N-biose and has important economic values and social benefits. At the same time, the synthetic method is efficient, mild, simple, easy to operate, low in cost and suitable for industrial production, and has a high practical application value.

The present application contains a Sequence Listing that has been submitted electronically in ASCII format and is hereby incorporated by reference in its entirety. The ASCII copy, created on Nov. 30, 2021, is named Substitute Sequence Listing_ST25.txt and is 16,384 bytes in size.

### BACKGROUND

### Technical Field

The present invention belongs to the technical field of bioengineering and oligosaccharide synthesis, and specifically relates to a method for synthesizing lacto-N-biose.

### Related Art

The background information is provided only to increase the understanding of the overall background of the present invention, but is not necessarily regarded as an acknowledgement or in any form suggesting that the information constitutes the prior art known to a person of ordinary skill in the art.

Breast milk, as the only material and energy link between the newborn and the mother after birth, is considered as the gold standard for infant nutrition. In addition to nutrients such as protein, fat and carbohydrates necessary for infant growth, what the breast milk provides also includes active components such as enzymes, antibodies, growth factors and oligosaccharides that can promote infant health and development. It is found that the biggest difference between cow and goat milk and breast milk lies in that breast milk is rich in functional human milk oligosaccharides (HMOs) and derivatives thereof with the content as high as 12-24 g/L, which are the third most important nutrient in breast milk. HMOs have special effects on the human body, especially for the perfection of the digestive system, intestinal health and immune system of infants and young children. Recently researchers found that HMOs can act as prebiotics for intestinal probiotics to regulate and promote the maturation of the intestinal immune barrier of infants, also can act as decoy molecules for pathogens, have antibacterial and antifungal effects, and protect infants from pathogen infection. Therefore, researches on HMOs are of great significance to the health of infants and young children, and addition of HMOs into milk powder to simulate breast milk components is particularly important for some infants who cannot be breast fed and the nutrition and health of infants after lactation.

However, due to limited availability and difficulty in large-scale synthesis with chemical methods, the research and wide application of biological functions of HMOs are restricted. In order to break through the bottleneck, it is necessary to establish a simple, efficient and economical synthetic route to obtain a large amount of the core structure lacto-N-biose (LNB, Galβ1-3GlcNAc, the chemical structural formula is as follows). Therefore, the low-cost, simple and easily separated technical process for biological synthesis of lacto-N-biose (LNB) was developed.

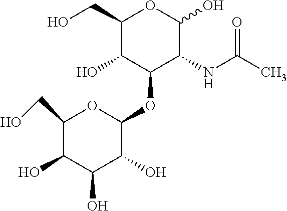 [SMILES: CC(=O)N[C@H]1C(O)O[C@H](CO)[C@@H](O)[C@@H]1O[C@@H]1O[C@H](CO)[C@H](O)[C@H](O)[C@H]1O]

At present, there have been a number of patents focused on biosynthesis technologies for production of human milk oligosaccharides in Escherichia coli, such as:

patent name: “A fucosyltransferase and genetic engineering bacteria and application thereof” (CN201611147477.8),

patent name: “Production of human milk oligosaccharides in a microbial host with modified input/output” (CN201680052611.8),

patent name: “A fucosyltransferase and application thereof” (CN201611147478.2), and

patent name: “A method for preparing lacto-N-neotetraose (LNNT) containing N-acetyllactosamine” (CN201510751641.5).

In all the above-mentioned patents, a technology of using Escherichia coli as a host for heterologous production of human milk oligosaccharides is adopted. However, it is a great challenge to remove Escherichia coli endotoxin in large-scale industrial production. Endotoxin is a component in the cell wall of gram-negative bacteria, also known as lipopolysaccharide, which is a substance that is toxic to the human body. However, food safety of human milk oligosaccharides, especially as an additive for infant milk powder, is obviously very important. Therefore, it is particularly important to find a synthetic method suitable for large-scale industrial production of human milk oligosaccharides with good safety, stable yield and high production efficiency.

### SUMMARY

In view of the above-mentioned shortcomings of the prior art, the present invention uses a multi-enzyme catalytic system with good biological safety and wide application, and an ATP regeneration cycle system is introduced into a multi-enzyme reaction system, so that the synthesis of lacto-N-biose and the utilization rate of substrates are improved. The present invention provides a novel lacto-N-biose synthetic route, which lays a foundation for large-scale industrial production of lacto-N-biose and has important economic values and social benefits.

In the first aspect, the present invention provides a method for synthesizing lacto-N-biose, and the method comprises:

adding galactokinase (GalK) and lacto-N-biose phosphorylase (LNBP) into a reaction system containing galactose (Gal), acetylglucosamine (GlcNAc) and lactose (Lac) as substrates to prepare lacto-N-biose; and

adding acetyl phosphate and acetate kinase (ACK) into the above reaction system for in-situ regeneration of ATP.

Further, the method further comprises: separating the product lacto-N-biose and ATP and ADP present in the system.

The synthesis principle of the present invention is as follows: in the present invention, a multi-enzyme catalytic system using galactose as a substrate is constructed to synthesize lacto-N-biose. The reaction substrate, galactose, is catalyzed by galactokinase to produce galactose-1-phosphate, and ATP is degraded into ADP at the same time; further, galactose-1-phosphate produces lacto-N-biose under the action of lacto-N-biose phosphorylase (LNBP). Under the action of acetate kinase (ACK), ADP obtained above combines with acetyl phosphate to regenerate ATP, realizing regeneration of ATP. In the present invention, only a low concentration of ATP needs to be put into reactants, the reaction cost is greatly reduced, the conversion efficiency of substrates is improved, and the effect of inhibiting galactokinase by a high concentration of ATP is avoided, thereby achieving complete conversion of substrates and shortening the reaction time.

In the second aspect, the present invention provides lacto-N-biose prepared and synthesized by the synthetic method. In addition, based on the synthetic method of the present invention, the synthesis of all oligosaccharides or polysaccharides containing and/or using lacto-N-biose as a skeletal structure is also within the protection scope of the present invention.

### Beneficial Technical Effects of the Present Invention

In the present invention, galactose is used as a raw material substrate, and an oligosaccharide structure catalytically synthesized in two steps includes lacto-N-biose. At the same time, all oligosaccharides or polysaccharides with lacto-N-biose as a skeletal structure can also be synthesized, the product preparation cost is low, and the practical value is high. In the present invention, the multi-enzyme catalytic system with good biological safety and wide application is used, and an ATP regeneration cycle system is introduced into the multi-enzyme reaction system, a simplified and economical coupling system for enzymatic synthesis of lacto-N-biose and regeneration of ATP is established, and the synthesis of lacto-N-biose and the utilization rate of substrates are improved.

In summary, the present invention provides a novel lacto-N-biose synthetic route, which lays a foundation for large-scale industrial production of lacto-N-biose and analogues thereof and has important economic values and social benefits. At the same time, the raw materials used in the present invention are easily available, the synthetic method is efficient, mild, simple, easy to operate, low in cost, environmentally friendly and suitable for industrial production, and has a high practical application value.

### BRIEF DESCRIPTION OF THE DRAWINGS

The accompanying drawings constituting a part of the present invention are used to provide a further understanding of the present invention. The exemplary examples of the present invention and descriptions thereof are used to explain the present invention, and do not constitute an improper limitation of the present invention.

FIG. 1 is a schematic diagram showing catalytic production of lacto-N-biose in two steps with galactose as a substrate of the present invention.

FIG. 2 is a synthetic comparison diagram of lacto-N-biose at an initial amount of 5 mM ATP after introduction of ATP regeneration cycle in Example 2 of the present invention.

FIG. 3 is a synthetic comparison diagram of lacto-N-biose at an initial amount of 7.5 mM ATP after introduction of ATP regeneration cycle in Example 3 of the present invention.

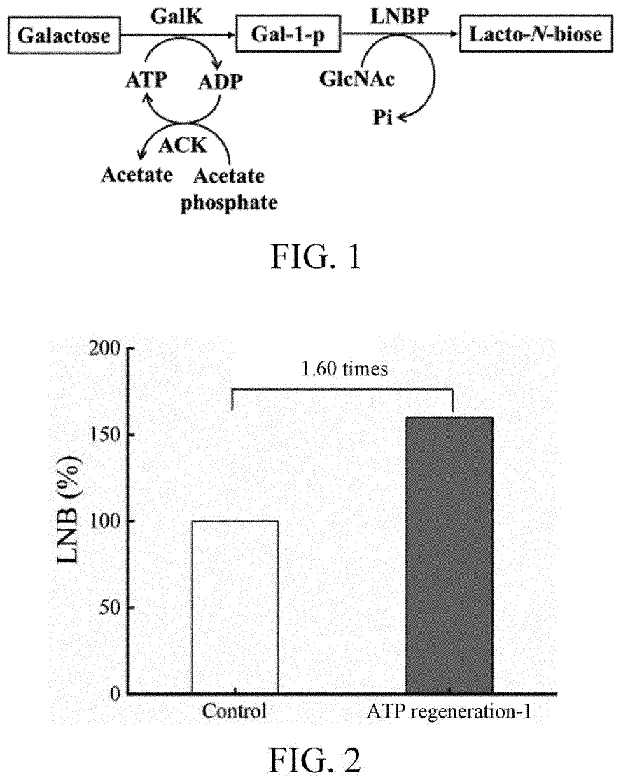

### DETAILED DESCRIPTION

It should be noted that the following detailed descriptions are all exemplary and are intended to provide a further description of the present invention. Unless otherwise specified, all technical and scientific terms used herein have the same meaning as commonly understood by those of ordinary skill in the art to which the present invention belongs.

It should be noted that terms used herein are only for the purpose of describing specific implementations and are not intended to limit the exemplary implementations of the present invention. As used herein, the singular form is intended to include the plural form, unless the context clearly indicates otherwise. In addition, it should be further understood that terms “comprise” and/or “include” used in this specification indicate that there are features, steps, operations, devices, assemblies, and/or combinations thereof. It should be understood that the protection scope of the present invention is not limited to the following specific implementation solutions. It should further be understood that terms used in the embodiment of this application are only for describing the specific implementation solutions, and are not intended to limit the protection scope of the present invention. In the following specific embodiments, if specific conditions are not indicated, experimental methods usually follow conventional methods and conditions of molecular biology in the art, and such techniques and conditions are fully explained in the literature. For example, Sambrook et al., techniques and conditions in “Molecular Cloning: Laboratory Manual” or conditions recommended by manufacturers are taken as references.

As mentioned earlier, it is currently difficult to synthesize human milk oligosaccharides on a large scale using chemical methods, and when a biotechnology is used for heterogeneous production of human milk oligosaccharides, the production efficiency is generally low, and food safety problems are likely to be caused.

In view of this, in a specific embodiment of the present invention, a method for synthesizing lacto-N-biose is provided and includes:

adding galactokinase and lacto-N-biose phosphorylase into a reaction system containing galactose, acetylglucosamine and lactose as substrates to prepare lacto-N-biose; and

adding acetyl phosphate and acetate kinase into the above reaction system for in-situ regeneration of ATP. The present invention aims at consuming one molecule of ATP energy in a galactokinase catalytic step, and constructs but is not limited to realization of ATP energy regeneration in acetyl phosphate with acetate kinase. Experimental results of the present invention prove that by introducing energy regeneration cycle, regeneration cycle of ATP and full conversion of substrates can be realized, thereby effectively improving the synthesis efficiency of lacto-N-biose.

In another specific embodiment of the present invention, the method further includes: separating the product lacto-N-biose and ATP and ADP present in the reaction system.

In the present invention, sources of enzymes used are not particularly limited, which may be extracted from natural bacteria, yeast or fungi and other microorganisms, produced by genetically engineered bacteria through genetic recombination and extracted from natural plant tissues or animal tissues. The enzyme product forms are also not particularly limited, which may be solid, powder, liquid, or an immobilized enzyme fixed on a carrier by a physical or chemical method. Enzymes may be commercially available products, or self-made products by enterprises or individuals.

In another specific embodiment of the present invention, the galactokinase, lacto-N-biose phosphorylase (LNBP) and acetate kinase are all produced by genetically engineered bacteria through genetic recombination.

Specifically, the method of the production includes: cloning expression vectors derived from galactokinase, lacto-N-biose phosphorylase and acetate kinase respectively; and obtaining target enzyme proteins by culturing and inducing hosts of the corresponding expression vectors.

In another specific embodiment of the present invention, the expression vectors are any one or more of viral vectors, plasmids, phages, phagemids, cosmids, F cosmids, bacteriophages or artificial chromosomes; viral vectors may include adenovirus vectors, retroviral vectors or adeno-associated virus vectors, and artificial chromosomes include bacterial artificial chromosomes (BAC), phage P1 derived carriers (PAC), yeast artificial chromosomes (YAC) or mammalian artificial chromosomes (MAC); further preferably, the expression vectors are plasmids; more preferably, the expression vectors are pET-28a plasmids.

In another specific embodiment of the present invention, the hosts include but are not limited to bacteria, fungi and eukaryotic cells, and are further selected from Escherichia coli, Bacillus, Bacillus subtilis, Saccharomyces cerevisiae, Trichoderma reesei and Penicillium oxalicum; more preferably, the hosts are Escherichia coli BL21 (DE3).

In another specific embodiment of the present invention, the method of the production includes: cloning expression plasmids pET28a-galk, pET28a-Lnbp and pET28a-ack derived from galactokinase, LNBP and acetate kinase respectively; and obtaining target enzyme proteins by culturing and inducing BL21(DE3) strains of the corresponding expression plasmids and carrying out purification (preferably by a nickel column).

Galactokinase source strains include but are not limited to Escherichia coli; LNBP source strains include but are not limited to Bifidobacterium; and ACK source strains include but are not limited to Escherichia coli.

In another specific embodiment of the present invention,

the amino acid sequence of galactokinase is shown as SEQ ID No. 1;

the amino acid sequence of LNBP is shown as SEQ ID No. 2; and

the amino acid sequence of acetate kinase is shown as SEQ ID No. 3.

In another specific embodiment of the present invention, the reaction system further contains ATP, and further, the concentration of ATP is 5-15 mM (preferably 7.5 mM).

The reaction system further contains magnesium ions (preferably MgCl₂) and a Tris-HCl buffer.

In another specific embodiment of the present invention, the concentration of MgCl₂ is 1-10 mM (preferably 3 mM); and the concentration of the Tris-HCl buffer is 10-200 mM (preferably 100 mM).

In another specific embodiment of the present invention, the reaction temperature of the reaction system is 25-45° C., and the reaction pH is 5.8-7.5.

In another specific embodiment of the present invention, the concentrations of the galactose and acetylglucosamine substrates are both 10-20 mM.

In another specific embodiment of the present invention, the concentration of galactokinase is 1-10 U/mL.

In another specific embodiment of the present invention, the enzyme concentration of LNBP is 100-300 U/mL.

In another specific embodiment of the present invention, the concentration of acetate kinase required for in-situ regeneration of ATP is 1-10 U/mL, and the concentration of acetyl phosphate is 2.5-5 mM.

In another specific embodiment of the present invention, lacto-N-biose synthesized by the synthetic method is provided. In addition, based on the synthetic method of the present invention, the synthesis of all oligosaccharides or polysaccharides with lacto-N-biose as a skeletal structure is also within the protection scope of the present invention.

The following further explains and describes the present invention through specific embodiments, but does not constitute a limitation on the present invention. It should be understood that these embodiments are only for illustrating the present invention and are not intended to limit the scope of the present invention. An experimental method without indicating a specific condition in the following embodiments is generally performed according to a conventional condition.

### Example 1: Synthesis of Lacto-N-Biose by Using Galactose as a Substrate

10 mM galactose, 10 mM ATP, 10 mM GlcNAc, 3 mM MgCl₂, 100 mM Tris-HCl, 5 U Galk and 168 U lacto-N-biose phosphorylase are added into a 1 mL reaction system. As shown in Table 1, Control is a control group without addition of lacto-N-biose phosphorylase (LNBP). After a reaction at 37° C. for 12 hours, the reaction system is boiled for 5 minutes and centrifuged, a supernatant is sampled, filtered through a filtering membrane and detected through a Biorad-HPX column, RID detection of corresponding substrates and products is carried out, reaction detection map (HPLC) comparison results verify the synthesis of lacto-N-biose.

TABLE 1
A reaction table showing catalytic production of lacto-N-biose in two
steps with galactose as a substrate

| | Gal | ATP | GalK | MgCl₂ | LNBP | GlcNAc |
|---|---|---|---|---|---|---|
| | mM | mM | U/mL | mM | U/mL | mM |
| Control | 10 | 10 | 5 | 3 | | 10 |
| 1 | 10 | 10 | 5 | 3 | 168 | 10 |

### Example 2: Synthesis of Lacto-N-Biose by Introducing ATP Regeneration Cycle

10 mM galactose, 7.5 mM ATP, 2.5 mM acetyl phosphate, 10 mM GlcNAc, 3 mM MgCl₂, 100 mM Tris-HCl, 5 U Galk, 3 U ACK and 168 U LNBP are added into a 1 mL reaction system. As shown in Table 2, the initial concentration of ATP in Control is 10 mM, and an ATP cyclic reaction is not introduced into the reaction system. After a reaction at 37° C. for 12 hours, the reaction system is boiled for 5 minutes and centrifuged, a supernatant is sampled, filtered through a filtering membrane and detected by a Biorad-HPX column, and RID detection of corresponding substrates and products is carried out. As shown in FIG. 2, under ATP-1 reaction conditions, the synthesis of LNB is increased by 1.60 times in comparison with the control group (without addition of ATP).

### Example 3

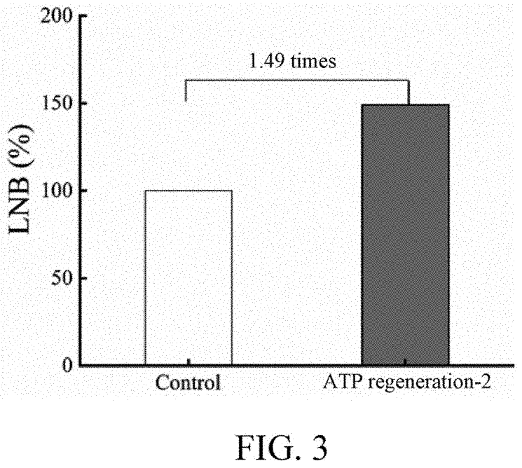

10 mM galactose, 5 mM ATP, 5 mM acetyl phosphate, 10 mM GlcNAc, 3 mM MgCl₂, 100 mM Tris-HCl, 5 U Galk, 3 U ACK and 168 U LNBP are added into a 1 mL reaction system. As shown in Table 2, the initial concentration of ATP in Control is 10 mM, and an ATP cyclic reaction is not introduced into the reaction system. After a reaction at 37° C. for 12 hours, the reaction system is boiled for 5 minutes and centrifuged, a supernatant is sampled, filtered through a filtering membrane and detected by a Biorad-HPX column, and RID detection of corresponding substrates and products is carried out. As shown in FIG. 3, under ATP-2 reaction conditions, the synthesis of LNB is increased by 1.49 times in comparison with the control group (without addition of ATP).

TABLE 2
A reaction table showing catalytic production of lacto-N-biose in two steps
with galactose as a substrate after introduction of ATP regeneration cycle

| | Gal | ATP | GalK | MgCl₂ | LNBP | GlcNAc | | ACK |
|---|---|---|---|---|---|---|---|---|
| Reaction | mM | mM | U | mM | U | mM | Acetyl-ACP | U |
| Control | 10 | 10 | 5 | 3 | 168 | 10 | | |
| ATP-1 | 10 | 7.5 | 5 | 3 | 168 | 10 | 2.5 | 3 |
| ATP-2 | 10 | 5 | 5 | 3 | 168 | 10 | 5 | 3 |

It should be noted that the foregoing embodiments are merely intended for describing the technical solutions of the present invention other than limiting the present invention. Although the present invention is described in detail with reference to the foregoing embodiments, a person skilled in the art may make modifications or equivalent substitutions to the technical solutions of the present invention as required, without departing from spirit and scope of the technical solutions of the present invention.

1. A method for synthesizing lacto-N-biose, the method comprising:
adding galactokinase and lacto-N-biose phosphorylase into a reaction system containing galactose, acetylglucosamine and lactose as substrates to prepare lacto-N-biose; and
adding acetyl phosphate and acetate kinase into the above reaction system for in-situ regeneration of ATP.

2. The method for synthesizing lacto-N-biose according to claim 1, wherein the method further comprises: separating the product lacto-N-biose and ATP and ADP present in the reaction system.

3. The method for synthesizing lacto-N-biose according to claim 1, wherein the galactokinase, lacto-N-biose phosphorylase and acetate kinase are all produced by genetically engineered bacteria through genetic recombination.

4. The method for synthesizing lacto-N-biose according to claim 3, wherein the method of the production comprises: cloning expression vectors derived from galactokinase, lacto-N-biose phosphorylase and acetate kinase respectively; and obtaining target enzyme proteins by culturing and inducing hosts of the corresponding expression vectors.

5. The method for synthesizing lacto-N-biose according to claim 1, wherein
the amino acid sequence of galactokinase is shown as SEQ ID No. 1;
the amino acid sequence of lacto-N-biose phosphorylase is shown as SEQ ID No. 2; and
the amino acid sequence of acetate kinase is shown as SEQ ID No. 3.

6. The method for synthesizing lacto-N-biose according to claim 1, wherein the reaction system further contains ATP, and the concentration of ATP is 5-15 mM.

7. The method for synthesizing lacto-N-biose according to claim 6, wherein the concentration of ATP is 7.5 mM.

8. The method for synthesizing lacto-N-biose according to claim 1, wherein the reaction system further contains MgCl₂ and a Tris-HCl buffer;
the concentration of MgCl₂ is 1-10 mM; and the concentration of the Tris-HCl buffer is 10-200 mM.

9. The method for synthesizing lacto-N-biose according to claim 8, wherein the concentration of MgCl₂ is 3 mM, and the concentration of the Tris-HCl buffer is 100 mM.

10. The method for synthesizing lacto-N-biose according to claim 1, wherein the reaction temperature of the reaction system is 25-45° C., and the reaction pH is 5.8-7.5.

11. The method for synthesizing lacto-N-biose according to claim 1, wherein the concentrations of the galactose and acetylglucosamine substrates are both 10-20 mM;
the concentration of galactokinase is 1-10 U/mL;
the enzyme concentration of LNBP is 100-300 U/mL;
the concentration of acetate kinase is 1-10 U/mL; and
the concentration of acetyl phosphate is 2.5-5 mM.

12. Lacto-N-biose synthesized by the method according to claim 1.


SEQUENCE LISTING (3 sequences)

SEQ ID NO: 1 - protein, 382 aa, Artificial Sequence
  Galactokinase
MSLKEKTQSLFANAFGYPATHTIQAPGRVNLIGEHTDYNDGFVLPCAIDYQTVISCAPRDDRKVRVMAADYENQLDEFSLDAPIVAHENYQWANYVRGVVKHLQLRNNSFGGVDMVISGNVPQGAGLSSSASLEVAVGTVLQQLYHLPLDGAQIALNGQEAENQFVGCNCGIMDQLISALGKKDHALLIDCRSLGTKAVSMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGDKGGVRMTGGGFGGCIVALIPEELVPAVQQAVAEQYEAKTGIKETFYVCKPSQGAGQC

SEQ ID NO: 2 - protein, 751 aa, Artificial Sequence
  Lacto-N-biose phosphorylase
MTSTGRFTLPSEENFAEKTKELAELWGADAIRNSDGTHLDESVLALGKKIYSAYFPTRAHNEWITLHMDETPQVYLLTGRVLAEADIVDVPLMDGFFEEQLKPNRDADPHKYWEVVDRTTNEVVDASLWTLDEDTDTVHVSGATPMHEYTVSFLAYIIWDPVEMYNHLTNGWGDKEHEIPFDIYHPATRKFVFDTFEQWLKDNPQVDVVRFTTFFYQFTLLFDQKQREKVVDWFGCACTVSPAALDDFEKEYGYRLRPEDFVDGGAYNSAWRVPRKAQRDWIDFLSGFVRANVKKLADMSHEAGKEAMMFLGDQWIGTEPYKDGFEDLGLDAVVGSIGDGTTTRMIADIPGVKYTEGRFLPYFFPDTFYEGNDPSIEGLDNWRKARRAILRSPISRMGYGGYLSLAAKFPKFVDTVTHIADEFRDIHDRTGGVAAEGELNVAILNSWGKMRSWMAFTVAHALPNKQTYSYYGILESLSGMRVNVRFISFDDVLEHGVADDIDVIINGGPVDTAFTGGDVWKNPKLTETLRAWVRGGGAFVGVGEPSSLARFQAGRFFQLADVLGVDEERYQTLSVDKYFPTVTPEHFITADVHVDPAAREAWEKAGYRIPLSGCGGGQGIKPLGGIDFGEPVANTFPVNEDVTLLRADGGQVQLAVNEYGKGRGVYISGLPYSAANARLLERALFWASHNEDKYTAYSSTNPECEVAVFPDAGQYCVINNTDRPQSTDVALPDGSVEHFDLDQSAIAWRNL

SEQ ID NO: 3 - protein, 400 aa, Artificial Sequence
  Acetate kinase
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA